(19) United States
(12) Patent Application Publication
(10) Pub. No.: US 2005/0129642 A1
(43) Pub. Date: Jun. 16, 2005

(54) Method of preventing undesired skin conditions beneath a woman's breasts

(76) Inventors: Judy C. Blanchard, Iola, WI (US); Kenneth H. Blanchard, Iola, WI (US)

Correspondence Address: DONALD R. SCHOONOVER, 4211 ROLLING HILLS DRIVE, NIXA, MO, 65714-8771, US

(21) Appl. No.: 10/733,520

(22) Filed: Dec. 12, 2003

(51) Int. Cl. A61K 7/32

(52) U.S. Cl. 424/65

(57) ABSTRACT

A method of preventing undesired skin conditions beneath a woman's breasts includes applying antiperspirant directly to the woman's skin beneath her breasts. The antiperspirant can be in gel form or in powder form or the like.

### BACKGROUND OF THE INVENTION

1. Field of the Invention

The present invention relates to the general art of hygiene, and to the particular field of antiperspirants for women.

2. Discussion of the Related Art

Perspiration is the body's method of preventing its temperature from rising above normal. The process of perspiring involves the secretion of sweat through pores in the skin. Sweat is ninety-nine percent water and as this water evaporates, it cools the body. The amount of sweat the body produces does not depend solely upon body temperature. Some individuals naturally perspire more than others. Exercise, anxiety, or mental stress can also increase the amount of perspiration that an individual experiences.

Despite the amount an individual perspires, when the perspiration rests on an area of skin that frequently makes contact with another area of skin, chaffing, rashes, or even infections can result. These forms of skin irritations are commonly experienced among women. While there are many forms of antiperspirants that can be used under one's arms, other areas of the body also are subject to this problem, and are generally overlooked by the antiperspirant art.

For example, women with large breasts experience this problem and the skin under their breasts can become greatly irritated by any amount of perspiration and any number of skin problems may occur as a result.

Therefore, there is a need for an antiperspirant that can be used by women in the area beneath their breasts.

The art does contain pads and bands as well as specially made brassiers that are intended to absorb moisture. Some women even place cloth beneath their breasts to absorb perspiration. However, cloths may extend out from under their breasts, and pads and the like may be uncomfortable as well as cumbersome. This will discourage women from using such devices. Furthermore, such devices may add to the expense of the clothing.

Therefore, there is a need for an antiperspirant that is not bulky, uncomfortable and inconvenient to use.

Therefore, there is also a need for an antiperspirant that is inexpensive to use.

### PRINCIPAL OBJECTS OF THE INVENTION

It is a main object of the present invention to provide an antiperspirant that can be used by women in the area beneath their breasts.

It is another object of the present invention to provide an antiperspirant that is not bulky, uncomfortable and inconvenient to use.

It is another object of the present invention to provide an antiperspirant that is inexpensive to use.

### SUMMARY OF THE INVENTION

These, and other, objects are achieved by a method which includes a step of applying antiperspirant directly to the skin beneath a woman's breasts. The antiperspirant can be in the form of a roll-on stick, a powder or the like and can be any form of chemical composition that is known in the antiperspirant art.

Using the method embodying the present invention will permit a woman to apply antiperspirant directly to the area beneath her breasts whereby the above-discussed problems will be avoided without the need for physical devices such as pads or the like.

### BRIEF DESCRIPTION OF THE DRAWING FIGURES

FIG. 1 is a perspective view of a first form of a container/applicator used in the method of applying antiperspirant to women's skin beneath her breasts embodying the present invention.

FIG. 2 is a perspective view of a second form of a container/applicator used in the method of applying antiperspirant to women's skin beneath her breasts embodying the present invention.

FIG. 3 is an elevational view taken along line 3-3 of FIG. 2.

FIG. 4 is a perspective view of a third form of a container/applicator used in the method of applying antiperspirant to a woman's skin beneath her breasts embodying the present invention.

### DETAILED DESCRIPTION OF THE PREFERRED EMBODIMENT

Other objects, features and advantages of the invention will become apparent from a consideration of the following detailed description and the accompanying drawings.

Referring to the Figures, it can be understood that the present invention is embodied in a method of protecting a woman's skin beneath her breasts. The method includes providing an antiperspirant in an applicator, and using the applicator and applying antiperspirant directly to the skin beneath the woman's breasts. The antiperspirant can be in gel form, powder form, or any other form that is convenient to the use of antiperspirants. The antiperspirant can be heat-activated or the like as is also known to those skilled in the art.

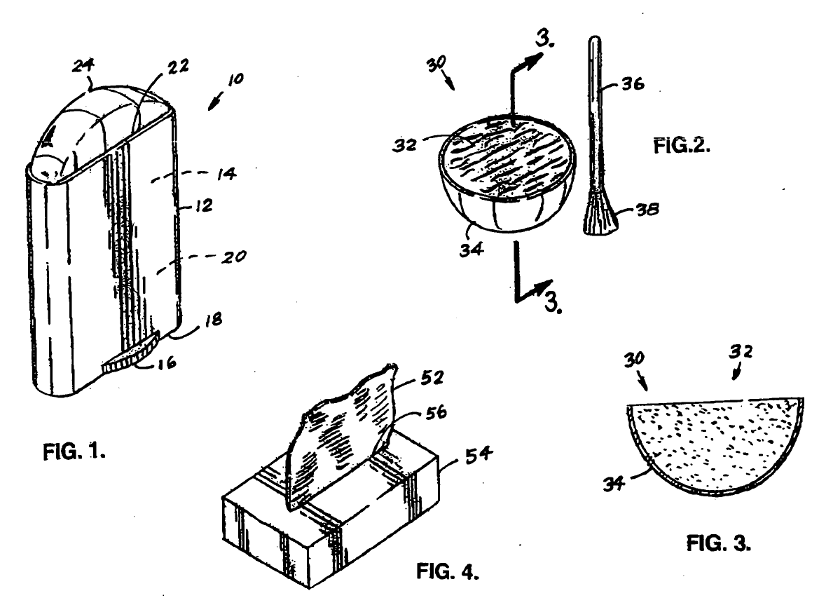

As shown in FIG. 1, a first form of antiperspirant applicator 10 is used to apply antiperspirant in gel form to a woman's body. Applicator 10 includes a housing 12 which can be constructed of plastic and be in the shape of a tube 14. Container 14 includes a rotatable knob 16 located near bottom edge 18 of the container 14. Applicator 10 operates in a manner known to those skilled in the art whereby rotation of knob 16 advances antiperspirant 20 out of opening 22 so that distal end 24 of the antiperspirant 20 is in position to be placed on the user's skin.

A second form of applicator is shown in FIG. 2 as applicator 30. Applicator 30 is used to apply antiperspirant in powder form to a user's skin. Thus, powder 32 is contained in a bowl-shaped housing 34, and the powder 32 can be of a form and composition known to those skilled in the art. Applicator 30 also includes a brush 36 having bristles 38 which are dipped into the powder 32 and then brushed onto the user's skin.

Applicator 50 is a third form of the applicator as shown in FIG. 4 and includes a towelette 52 that is saturated with an antiperspirant material. The towelettes 52 are stored in a container 54 in a manner known to those skilled in the art and are dispensed one by one through a top opening 56.

It is understood that while certain forms of the present invention have been illustrated and described herein, it is not to be limited to the specific forms or arrangements of parts described and shown.

1. A method of preventing undesired skin conditions comprising:
(a) providing an antiperspirant in an applicator; and
(b) using the applicator and applying antiperspirant directly to the skin beneath a woman's breasts.

2. The method as described in claim 1 wherein the antiperspirant is in gel form.

3. The method as described in claim 1 wherein the antiperspirant is in powder form.
